(19) United States
(12) Patent Application Publication
(10) Pub. No.: US 2023/0309918 A1
(43) Pub. Date: Oct. 5, 2023

(54) TREATING PATHOGENIC INFECTIONS

(71) Applicants: Mark H. KRIETZMAN, Rolling Hill, CA (US); Renz Wolfgang, Rheinfelden (DE)

(72) Inventors: Mark H. Krietzman, Rolling Hills, CA (US); Wolfgang Renz, Rheinfelden (DE)

(21) Appl. No.: 18/023,336

(22) PCT Filed: Aug. 25, 2021
(86) PCT No.: PCT/US2021/047474

§ 371 Date: Feb. 24, 2023

Related U.S. Application Data
(60) Provisional application No. 63/069,998, filed on Aug. 25, 2020.
(60) Provisional application No. 63/070,116, filed on Aug. 25, 2020.

(51) Int. Cl. A61B 5/00; A61B 5/145; G16H 20/13; A61B 5/0205

(52) U.S. Cl. CPC A61B 5/4839; A61B 5/02055; A61B 5/14532; G16H 20/13

(57) ABSTRACT

Controllers induce a hypoglycemic condition within a predetermined blood glucose range and bacterial or viral infection are targeted for death. One or more controllers are in signal communication with sensors and measure physiological vital signs. Controllers are in signal communication with one or more fluid flow control devices to control delivery of at least insulin and glucose and at least one cocktail containing at least one of an antibiotic and an antiviral. The fluid control devices are in signal communication with at least one microprocessor having memory and the one or more physiological sensors, one or more databases or lookup tables and, wherein the controller controls the fluid control devices for at least insulin glucose, and the cocktail to keep blood glucose level (BGL) within a target hypoglycemic range.

### CROSS REFERENCE TO RELATED APPLICATIONS

The present application is a 371 National Stage of International Patent Application No. PCT/US2021/047474, filed Aug. 25, 2021, which claims priority to U.S. Provisional Pat. Application Serial No. 63/069,998, filed Aug. 25, 2020, entitled METHOD OF TREATMENT FOR PULMONARY INFLAMMATION; and also claims priority claims priority to U.S. Provisional Pat. Application Serial No. 63/070,116, filed Aug. 25, 2020, entitled METHOD OF TREATMENT FOR INFLAMMATION, which are hereby incorporated by reference in their entirety.

### FIELD OF THE DISCLOSURE

Systems and methods to control hypoglycemic conditions in a primate during therapeutic treatment of pathogens.

### BACKGROUND

Lyme disease is caused by the bacterium Borrelia Burgdorferi which is an extracellular pathogen and is transmitted to humans through the bite of infected back legged ticks. Lyme disease, is the most commonly reported vector-borne disease in the United States-with 30,000 cases being reported to the CDC annually and at least two studies conducted by the CDC concluded due to under reporting the annual cases are estimated 300,000 individuals are infected with Lyme disease each year in the U.S. Associated medical costs of initial treatment and extended healthcare for ongoing symptoms attributed to post-treatment Lyme disease syndrome are estimated to be between $712 million $1.3 billion each year. It causes inflammation and other symptoms. Lyme disease is extremely difficult to eradicate in primate hosts. Lyme borreliosis is the most common human tick-borne disease in the Northern hemisphere. Its prevalence is estimated to range between 20 and 100 cases per 100,000 people in the US and about 100 to 130 cases per 100,000 in Europe. The symptoms of Lyme disease can range from erythema migraines to more systematic disorders such as arthritis and neurological complications, termed neuroborreliosis. Manifestations of neuroborreliosis include radiculoneuritis, meningitis, and facial palsy. It is well-documented that B. burgdorferi is capable of penetrating into the central nervous system. It is believed that during infection Borrelia burgdorferi will induce an immune response conducive to the chemotaxis of immune cells and subsequently lead to a pro-inflammatory state. (Thompson D, Sorenson J, Greenmyer J, Brissette CA, Watt JA (2020) The Lyme disease bacterium, Borrelia burgdorferi, stimulates an inflammatory response in human choroid plexus epithelial cells. PLOS ONE 15(7): e0234993. https://doi.org/10.1371/journal.pone.0234993) . It is therefore a desideratum to reduce and remove the populations of bacterium Borrelia Burgdorferi from hosts.

### DISCLOSURE

Disclosed herein are aspects of devices, methods, and systems of maintaining a dynamically controlled hypoglycemic environment to reduce bacterial cell populations in a primate.

Disclosed herein are aspects of devices, methods, and systems of delivering a method of at least one of an effective treatment a supplement to bacterial cell populations, including but not limited to disrupting the cellular membrane of the bacterial cells including Borrelia Burgdorferi then contacting the bacteria with an effective amount of an antibiotic agents which is more likely to cellularly disrupted (cause apoptosis to) bacteria then normal cells.

Disclosed herein are aspects of devices, methods, and systems of reducing infection by bacteria or virus. In some exemplary implementations’ aspects include placing a primate hosts cells in hypoglycemic conditions to increased membrane permeability to at least one antibiotic compound which also can be referred to as “cocktail” or “cocktail components”.

In some exemplary implementations aspects include maintaining hypoglycemic conditions in a primate to increased membrane permeability of synovial membranes to allow passage of the antibiotics into the synovial space and / or fibroblast-like synoviocytes (FLSs), of the antibiotic or cocktail.

Disclosed herein are aspects of devices, methods and systems for drug development and testing under hypoglycemic conditions. The method further comprising delivering one of a supplement and a pharmaceutically effective dose of antibiotics to a population of bacterial in an animal model which has been selectively placed in a controlled hypoglycemic condition. In some cases, the animal model is a primate. In some cases, the animal model is a humanized non-primate, in some cases the animal model is a non-primate mammal.

Disclosed herein are aspects of devices, methods, and systems of delivering one of a supplement and a pharmaceutically effective dose of one or more antibiotics to a population of bacteria in a primate, the primate selectively placed in a controlled hypoglycemic condition whereby death of the bacteria occurs at a higher rate than death of the same bacteria under non hypoglycemic conditions.

Disclosed herein are aspects of devices, methods, and systems of delivering a phased or sequenced series of cocktail compounds, forming a pharmaceutically effective dose, to cause death in bacteria or viruses when the primate is under a controlled hypoglycemic conditions.

In the above exemplars one or more controllers control hypoglycemic conditions in the test animal or primate via data received from one or more sensor inputs whereby fluid control devices to control the flow of insulin, glucose and optionally additional cocktail component and adjuvants as well as oxygen.

Aspects of the delivery systems, control system and methods disclosed include a control system are configured to maintain a host in a controlled hypoglycemic condition and automatically adjust. Condition to maintain the hypoglycemic condition within a target range of blood glucose levels (BGL) and above a first threshold. In some instances, the system includes logic to raise BGL when the first threshold or a lower second threshold is reached. The thresholds and ranges may be personalized based on collected individual data about a patient prior to treatment with the hypoglycemic method disclosed herein. The system and method include, but are not limited to:
(i) measuring the normal level of the host’s insulin pretreatment over a period of between 15 minutes and 48 hours before administering the hypoglycemic protocols.
(ii) Preparing a host specific algorithm in the form of computer code stored in memory and configured to be used in a microprocessor in signal communication with one or more controllers configured to adjust insulin delivery to maintain a hypoglycemic state in the host during therapeutic administration.
(iii) monitoring with sensors in signal communication to the one or more controller one or more of the host’s vital signs, including but not limited to, heart rate (HR), blood pressure (BP), electrocardiogram (EKG), electroencephalogram (EEG), oxygen saturation (O₂), galvanic skin response (GSR), skin moisture, pupillary dilation (PD), temperature (T), respiration (R) rate, and blood glucose level (BGL).
(iv) using a controller to meter out at least boluses of insulin (via one or more devices) to place the host in a temporarily hypoglycemic condition at one of a predetermined target range and above a predetermined hypoglycemic threshold.
(v) using a controller to meter out at least boluses of glucose (via one or more devices) to keep the host above a predetermined hypoglycemic threshold.
(vi) The controller(s) configured to use sensor data to control at least one of control the amount and the rate of insulin delivery to keep host to maintain blood glucose levels (BGL) within a defined range corresponding to the target hypoglycemic condition for the host. In some instances, the target hypoglycemic condition for the host is related to or arise from the previously measured levels for that host. In some instances, if measured oxygen saturation is below a predetermined level the controller administers additional to the host.
(vii) optionally one or more alarms are generated via the controller(s) if the controlled hypoglycemic conditions in the host (as measured by the system) are outside of a range selected for the host at a given time during treatment. The alarms may be any form including but not limited to visual, auditory, and haptic. The alarms may at least one of interrupt the insulin delivery, cause glucose to be delivered, cause oxygen to be delivered until vital signs are restored to within the target range.
(viii) Optionally pharmaceutically effective amounts of at least one of an antihistamine and an antiemetic may be administered prior to insulin delivery.
(ix) after the host is in the hypoglycemic condition the devices and systems sequence administration of pharmaceutically effective amounts of one or more antibiotic or antiviral agents in a pharmaceutically effective dose, under hypoglycemic conditions.
(x) optionally measuring at least one of the host’s vital signs, including but not limited to ECG, EKC, blood pressure, oxygen saturation, heart rate, galvanic skin response, skin moisture, and temperature response over a period of between 15 minutes and 48 hours before treatment and collecting said data.
(xi) optionally one or more alarms configured in the computer code are configured so that the controller(s) generates an alarm if measured vital sign(s) is outside a predefined range. In some instances, using the previously measured vital sign data to set said range. The alarms may at least one of interrupt the insulin delivery, cause glucose to be delivered, cause oxygen to be delivered until vital signs are restored to within the target range.

In some instances, prior to step (i) at a predetermined interval the host consumes a known quality sugar in a predetermined form with a Glycemic Index (GI) and a known glycemic load (GL). By supplying a consistent food type of a fixed quantity and with a known GI and GL the measurement of the host’s innate systems response to the consumed material can be measured via blood glucose monitoring, and used at least in part, as a data point to set the target range for hypoglycemic conditions for that host’s treatment.

In some instances, prior to step (iv) at a predetermined interval the host consumes a known quality sugar in a predetermined form with a known GI and GL. By supplying a food type of a fixed quantity and with a known GI and GL the controller can use look up tables (LUT) or refer to prior measurements of the host’s consumption of the same GI and GL food and used, at least in part, as a data point when maintaining the target range for hypoglycemic conditions for that host.

Disclosed herein are aspects of devices, methods, compositions of matter and systems to induce a hypoglycemic condition within a predetermined blood glucose range for treating infections including bacterial and viral cells (including but not limited to herpes) including one or more controllers in signal communication with at least a BGL and one or more sensors which measure an aspect that is physiological and in signal communication with one or more fluid flow control devices to control deliver of at least insulin and glucose and at least one cocktail containing at least one of antibiotic and antiviral components. The fluid control devices are in signal communication with at least one microprocessor having memory and the one or more physiological sensors, one or more databases or lookup tables and, wherein the controller controls the fluid control devices for at least insulin glucose, and the cocktail to keep blood glucose level (BGL) within a target hypoglycemic range for BGL for the patient. In some instances, the controller receives sensor data inputs and adjust the hypoglycemic target range for BGL in response to sensory data received. In some instances, the sensor data is one of BGL, oxygen saturation, heart rate, blood pressure, galvanic skin response, temperature, EEG, ECG, and pupillary response. In some instances, the controller controls the administration of at least one of oxygen and hydrogen.

Disclosed herein are aspects of devices, methods, compositions of matter and systems to induce a hypoglycemic condition within a predetermined blood glucose range for treating infections including bacterial and viral cells (including but not limited to herpes) including one or more controllers in signal communication with at least a BGL and one or more sensors which measure an aspect that is physiological and in signal communication with one or more fluid flow control devices to control deliver of at least insulin and glucose and at least one cocktail containing at least one of antibiotic and antiviral components. The fluid control devices are in signal communication with at least one microprocessor having memory and the one or more physiological sensors, one or more databases or lookup tables and, wherein the controller controls the fluid control devices for at least insulin glucose, and the cocktail to keep blood glucose level (BGL) within a target hypoglycemic range for BGL for the patient. In some instances, the controller receives sensor data inputs and adjust the hypoglycemic target range for BGL in response to sensory data received. In some instances, the sensor data is one of BGL, oxygen saturation, heart rate, blood pressure, galvanic skin response, temperature, EEG, ECG, and pupillary response. In some instances,, the cocktail is at least one of Quercetin, curcumin, vitamin C, clarithromycin, docycyclin, metronidazol. mezloxillian, piperacillin, azlocillin acylampicillin, amoxicillin, cefuroxime, Ceftriaxone, Acyclovir, Famciclovir, Valacyclovir and phenylbutyrate (PBA). In some instances, the infection is B. burgdorferi and the cocktail is at least one clarithromycin, docycyclin, metronidazol, mezloxillian, piperacillin, azlocillin acylampicillin, amoxicillin, cefuroxime, and Ceftriaxone. In some instances,, the active agents in the cocktail are each less than 50% the maximum tolerated dose (MTD). In some instances, the active agents in the cocktail are less than 50% the minimum effective dose (MED). In some instances, the active agents in the cocktail are less than 25% the minimum effective dose (MED).

1. A method to reduce infection in a primate, the method comprising:
inducing a hypoglycemic condition in a primate having infection within a target BGL range by way of infusion, controlled by a controller, of at least insulin;
monitoring vital signs of the primate with one or more sensors each of which monitor a physiological aspect of the primate and are in signal communication with the controller;
controlling with a controller infusion of a cocktail containing at least one of antibiotic and an antiviral components into the primate while the primate is in a hypoglycemic condition;
wherein the controller receives data inputs form the sensors and at least in part uses that input data to one of maintain the primate’s BGL within a hypoglycemic range for BGL and alter the hypoglycemic BGL range or lower threshold for the hypoglycemic BGL based on the received sensor data; and.
wherein cellular membranes of the primate are made more susceptible to the influx of cocktail components by way of the hypoglycemic condition.

2. The method to reduce infection in a primate, of claim 11 wherein the infection is one of HSV and B. burgdorferi.

3. The method to reduce infection in a primate, of claim 11 wherein the sensor data is one of BGL, oxygen saturation, heart rate, blood pressure, galvanic skin response, temperature, EEG, ECG, and pupillary response.

4. The method to reduce infection in a primate, of claim 11 wherein the controller controls the administration of at least one of oxygen and hydrogen.

5. The method to reduce infection in a primate, of claim 11 wherein the infection B. burgdorferi and the is cocktail is at least one of, clarithromycin, docycyclin, metronidazol. mezloxillian, piperacillin, azlocillin, acylampicillin, amoxicillin, cefuroxime, Ceftriaxone.

6. The method to reduce infection in a primate of claims 15 wherein the active agents in the cocktail are each less than 50% the maximum tolerated dose (MTD).

7. The method to reduce infection in a primate of claims 15 wherein the active agents in the cocktail are less than 50% the minimum effective dose (MED).

8. The method to reduce infection in a primate of claims 15 wherein the active agents in the cocktail are less than 25% the minimum effective dose (MED).

9. The method to reduce infection in a primate of claims 15 wherein the active agents in the cocktail are less than 15% the minimum effective dose (MED).

10. The method to reduce infection in a primate of claim 11 the method further comprising the controller raises BGL in the primate by way of infusion of at least glucose to maintain the hypoglycemic range for the primate’s BGL.

11. The method to reduce infection in a primate of claims 20 the method further comprising the controller administers magnesium before or during administration of glucose.

It is appreciated by those skilled in the art that some of the circuits, components, controllers, modules, and/or devices of the system disclosed in the present application are described as being in signal communication with each other, where signal communication refers to any type of communication and/or connection between the circuits, components, modules, and/or devices that allows a circuit, component, module, and/or device to pass and/or receive signals and/or information from another circuit, component, module, and/or device. The communication and/or connection may be along any signal path between the circuits, components, modules, and/or devices that allows signals and/or information to pass from one circuit, component, module, and/or device to another and includes wireless or wired signal paths. The signal paths may be physical such as, for example, conductive wires, electromagnetic wave guides, attached and/or electromagnetic or mechanically coupled terminals, semi-conductive or dielectric materials or devices, or other similar physical connections or couplings. Additionally, signal paths may be non-physical such as free-space (in the case of electromagnetic propagation) or information paths through digital components where communication information is passed from one circuit, component, module, and/or device to another in varying analog and/or digital formats without passing through a direct electromagnetic connection. These information paths may also include analog-to-digital conversions (“ADC”), digital-to-analog (“DAC”) conversions, data transformations such as, for example, fast Fourier transforms (“FFTs”), time-to-frequency conversations, frequency-to-time conversions, database mapping, signal processing steps, coding, modulations, demodulations, etc. The controller devices and smart devices disclosed herein operate with memory and processors whereby code is executed during processes to transform data, the computing devices run on a processor (such as, for example, controller or other processor that is not shown) which may include a central processing unit (“CPU”), digital signal processor (“DSP”), application specific integrated circuit (“ASIC”), field programmable gate array (“FPGA”), microprocessor, etc. Alternatively, portions DCA devices may also be or include hardware devices such as logic circuitry, a CPU, a DSP, ASIC, FPGA, etc. and may include hardware and software capable of receiving and sending information

### FIGURES

The disclosure may be better understood by referring to the following figures. The components in the figures are not necessarily to scale, emphasis instead being placed upon illustrating the principles of the invention. In the figures, like reference numerals designate corresponding parts throughout the different views.

FIG. 1 is a diagram of a host with sensor.

FIG. 2 is a diagram of a system overview

FIG. 3 is a flow diagram for the delivery system.

FIG. 4 illustrates aspects of a logic of the system logic.

FIG. 5 shows a nonexclusive list of chemical structures of analogs of Quercetin.

FIG. 6 shows the chemical structure of Curcumin.

FIGS. 7 & 8 are tables showing sequenced administration of hypoglycemic condition and infusion of therapeutic compounds.

All descriptions and callouts in the Figures and all content of any referenced citation are hereby incorporated by this reference as if fully set forth herein.

### FURTHER DISCLOSURE

The compositions disclosed herein can be included in a pharmaceutical or nutraceutical composition together with additional active agents, carriers, vehicles, excipients, or auxiliary agents identifiable by a person skilled in the art upon reading of the present disclosure, and such compositions are within the scope of this disclosure. All publications cited herein are hereby incorporated by reference as if fully set forth herein.

The pharmaceutical or nutraceutical compositions preferably comprise at least one pharmaceutically acceptable carrier. In such pharmaceutical compositions, the compositions disclosed herein form the “active compound,” also referred to as the “active agent.” As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds and/or adjuvants can also be incorporated into the compositions. A pharmaceutical composition is formulated to be compatible with its intended route of administration.

Administration″ and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, primate, dog, cat, and rabbit) and most preferably a human. Administration by inhalation, the gas or gases are delivered orally.

As used herein a “primate host” is defined to include a monkey, baboon, chimpanzee, gorilla, and a human. Nonhuman primates are appreciated to themselves be susceptible to infection by retroviruses and in particular immunodeficiency viruses and represent well-established animal models as to human response with an appreciation that physiological differences often require different doses in milligrams per kilogram for a nonhuman primate animal model relative to a human.

Administration″ and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, primate, dog, cat, and rabbit) and most preferably a human.

A pharmaceutically effective dose (ED) or effective concentration (EC) is a dose or concentration of an element (such as hydrogen), a phytochemical, compound ore drug that produces a biological response. The term effective dose is used when measurements are taken in vivo, while the term effective concentration is used when the measurements are taken in vitro. This is generally defined by the range between the minimum effective dose (MED) and the maximum tolerated dose (MTD). The MED is defined as the lowest dose level of a pharmaceutical product that provides a clinically significant response in average efficacy, which is also statistically significantly superior to the response provided by the placebo. Similarly, the MTD is the highest possible but still tolerable dose level with respect to a pre-specified clinical limiting toxicity. In general, these limits refer to the average patient population.

Cell senescence can be associated with a progressive and marked increased rate of glucose metabolism through glycolysis. Senescent cells display widespread changes in chromatin structure (referred to as senescence associated heterochromatin foci, SAHF) and gene expression profiles, which lead to highly active cellular metabolism and secretion of cytokines (TGF-β, IL-1a, -1β and -6), chemokines (IL-8, CXCL1), growth factors (FGF, HGF) and proteases (MMP-1, -3, and -13), collectively defined as senescence associated secretory phenotypes (SASP). SASP is a key factor that leads to the increase in senescence of populations of normal cells in proximity to senescent cells.

Plasma glucose levels are maintained within a narrow range by the pancreatic hormone’s glucagon and insulin. A normal level threshold for BGL is about 140 mg/dL Hypoglycemia, is general a BGL of below 50 mg/dl in a non-diabetic and it triggers secretion of glucagon by pancreatic α cells, which promotes glycogenolysis and gluconeogenesis in the liver, and lipolysis in adipose tissue. On the other hand, hyperglycemia triggers secretion of insulin from pancreatic β cells, which promotes glucose uptake for energy production and anabolic processes such as glycogen synthesis and lipogenesis in the liver, muscles, and adipose tissue.

We have observed that controlled hypoglycemic conditions applied to patients via an infusion of insulin during combined with administration of a cocktail is associated with preferential reduction in B. burgdorferi. The term cocktail refers to one or more of antibiotics and may include supplements and adjuvants. In some instances, the administration of the cocktail during controlled hypoglycemic conditions results in higher intercellular concentrations of cocktail therapeutics in cells then without hypoglycemic conditions. Many effective antibiotics are unsuccessful in penetrating the synovial membrane when administered at a MED and without hypoglycemic conditions approach of exceed their MTD. Our controlled hypoglycemic conditions are configured to reduce the MED whereby the cocktail is administered at below the MTD for cocktail components and can achieve effective therapeutic doses to reach bacteria within synovial membrane.

The system hardware, software, microprocessors, and controllers are configured to adjust the administration (rate and quantity) of insulin to maintain controlled hypoglycemic conditions for an individual patient based on previously collected patient data. The system hardware, software, microprocessors, and controllers may set off alarms if blood glucose is outside a patients predetermined range. The system hardware, software, microprocessors, and controllers are configured to control the infusion of glucose (rate and quantity), oxygen (rate and quantity) and cocktail components (rate and quantity of each) and set off alarms if measured blood glucose and/or vital signs are outside predetermined ranges or levels.

The system hardware, software, microprocessors, and controllers are configured to adjust the insulin administration to maintain controlled hypoglycemic conditions for an individual patient based on previously collected patient data which is used to define an individual target range for and define at least a first BGL lower threshold for that individual. In some instances, that data collected is also sued to define a second lower BGL (also known as an alarm level) for that individual.

The system hardware, software, microprocessors, and controllers may override the target for blood glucose target range or first or second threshold levels of a patient based on one or more inputs of sensor data. The microprocessor compares the patient sensor data being collected in real time during hypoglycemic conditions with one or more of a look up table based on human physiology, a look up table (LUT) based on measurements of the patient made prior to treatment, threshold level preset in a decisioning module, and if one or more sensor measurements exceed a risk level a target range or threshold limit may be altered and the controller will then administer an effective amount of insulin or glucose (for example) to raise or lower the BGL to the revised or altered target range or above a revised threshold. The system can override the continuation of insulin administration or reduce the amount given. The system can add glucose to the patient, the system can add oxygen to the patient and the system can adjust infusion of cocktail components to the patient. In some instances, an alarm will be set-off if the sensor data exceeds a threshold. The previous collection of patient data may be over 15 minutes or more but preferably over several hours or over a day.

A simplified overview is that an in vivo animal model having an infection of B. burgdorferi is given a bolus of insulin, the cellular insulin receptors (IR) activate expecting sugar instead the insulin is followed by cocktail components. Synoviocytes, express a large number of insulin receptors and under hypoglycemic conditions will allow for a higher concentration of cocktail components to enter the synovium then would enter when the primate blood glucose levels (GL) are at normal levels. B. burgdorferi DNA was detected in synovial fluid from 85% of the 88 patients with Lyme arthritis. (Nocton JJ et al. Detection of Borrelia burgdorferi DNA by polymerase chain reaction in synovial fluid from patients with Lyme arthritis. N Engl J Med 1994 330 229234).

A simplified overview is that an in vivo animal model having an infection of B. burgdorferi or herpes is given a bolus of insulin, the cellular insulin receptors (IR) activate expecting sugar instead the insulin is followed by cocktail components. Ganglion roots express insulin receptors and under hypoglycemic conditions will allow for a higher concentration of cocktail components to enter the root ganglia then would enter when the primate blood glucose levels (GL) are at normal levels. The ability of herpes simplex virus type 2 (HSV-2) to establish latency in and reactivate from sacral dorsal root sensory ganglia is the basis for recurrent genital herpes.

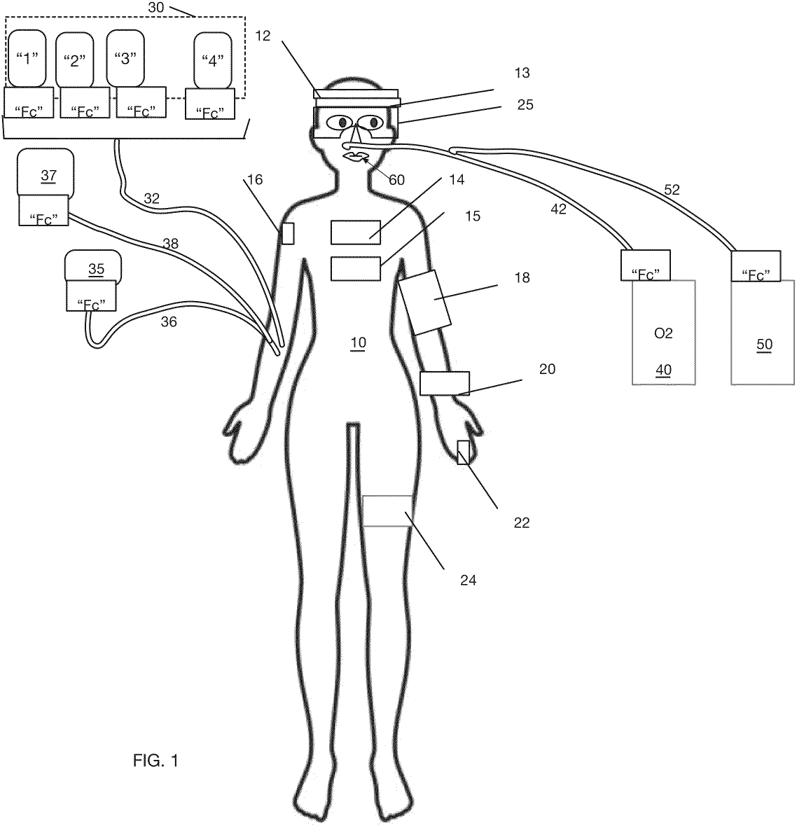

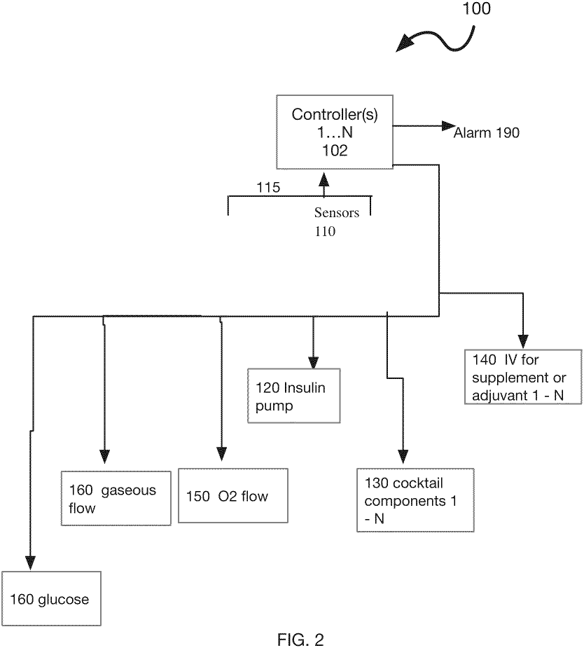

In another simplified overview set forth in FIGS. 1 and 2 in which a primate 10 is connected to sensors 110 which are in signal communication with a controller 102. The primate is connected to or measured by a multitude of sensors 110. Including but not limited to sensors to measure temperature 12, EEC 13, EKG 14, galvanic skin response (which measured sweatiness) 15, blood glucose levels (BGL) 16, blood pressure 18, heart rate 20, oxygen saturation 22 and additional measurements such as skin moisture which may include cortisol level measurements 24. Pupillary response is measured by machine vision optical system 25. Cocktail components 30 (1-N) each in a containment vessel are connected to the patient or host via fluid pathways and the fluid pathways each have a flow control device “fc” to start, stop and regulate the fluid flow, flow control devices include but are not limited to remotely controlled syringe pumps, peristaltic IV pumps, piston driven pumps and valves. Fluid control devices (“fc”) are in signal communication with one or more controllers wherein the fluid flow rate is controlled in response to microprocessor control which in turn is based at least in part on sensor data received and analyzed by the system processors.

The cocktails components are connected to the patient via a fluid communication pathway 32. An insulin source 35 in a containment vessel has a flow control device “fc” in signal communication with a controller and is connected to the patient via a fluid communication pathway 36. A glucose source 37 in a containment vessel has a flow control device “fc” in signal communication with a controller and is connected to the patient via a fluid communication pathway 38. An oxygen source 40 in a containment vessel has a flow control device “fc” in signal communication with a controller and is connected to the patient via a fluid communication pathway 42. A second gaseous fluid source 50 in a containment vessel has a flow control device “fc” in signal communication with a controller and is connected to the patient via a fluid communication pathway 52. The second gaseous fluid source includes but is not limited to hydrogen, oxyhydrogen, and vaporized or atomized cannabinoids. Oral ingestion via the mouth 60 may be an alternative for some of the cocktail components or optional compounds.

A control system overview 100 is a simplified diagram showing controllers 102 which are in signal communication 115 with the sensor 110 outputs. The controller processes the data from the sensors and decisions, based on LUTs, predetermined ranges for a patient and threshold levels to control insulin rate of administration and quantity. The controller also controls the administration of cocktail components 130. The controller 102 also controls the administration of supplement or adjuvant components 140. The controller 102 also controls the administration of oxygen 150. The controller also controls the flow of gaseous fluids 160 such as hydrogen, oxyhydrogen and/or cannabinoids. The controller 102 also controls the administration of glucose 37. The controller also triggers or sets alarms 190 for out of threshold or range measurements. Normally when blood glucose level reach less than 30 mg/dl the system will administer a controlled release of glucose, at a rate based on sensor data, and within a predetermined first threshold (or safe limit). The system is configured for the individual and to avoid a significant glucose deficit which may impair brain function. In those instances, wherein levels fall below a second threshold which is below the first threshold alarms 190 will signal the and the system can administer magnesium to allow for an increased rate of glucose infusion. If glucose is infused too fast physiological stress is created in the vein and cramping. By adding magnesium, a higher flow of glucose can be used to restore the patient above the second threshold. Post treatment after patient stabilized and has BGL above a premeasured and predetermined base level (generally about 15-20 minutes) an oral infusion of at least 250 cc Glucose 40% and also a glucose-based fluid or juice such Coke and/or Apple juice at 500 cc orally helps the patient to maintain blood glucose levels.

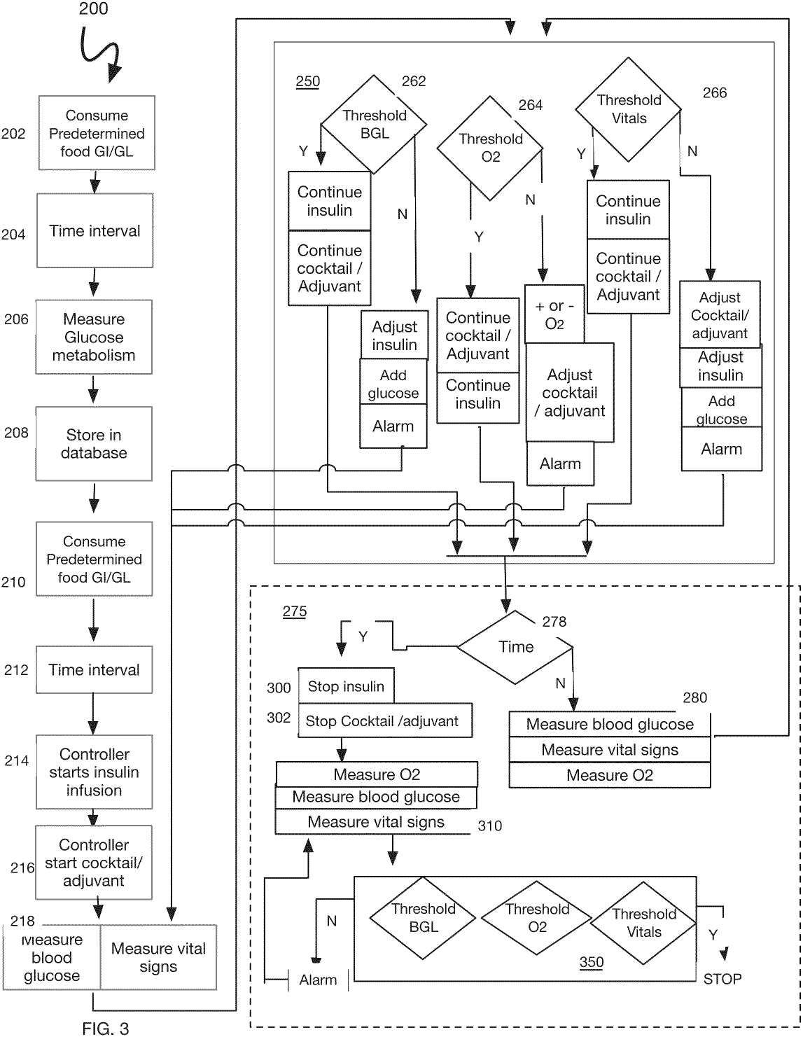

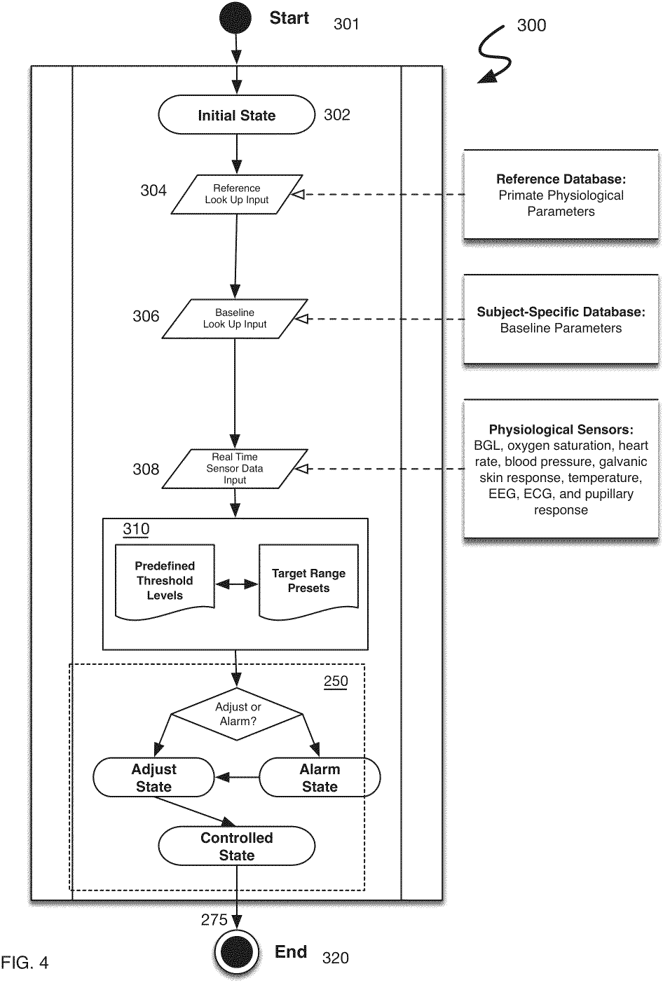

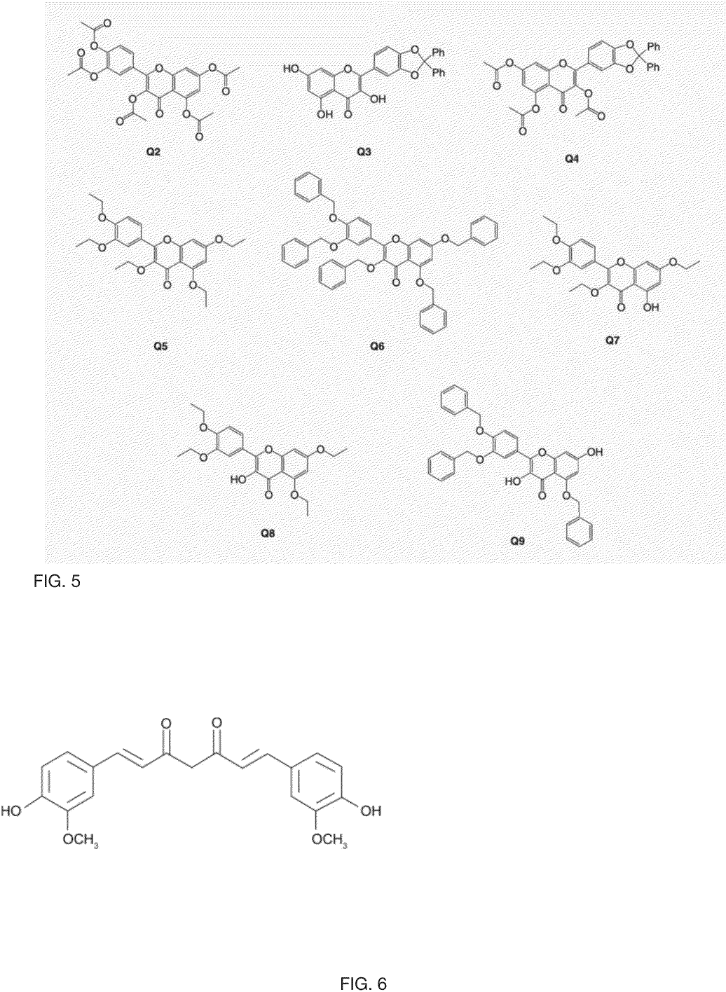

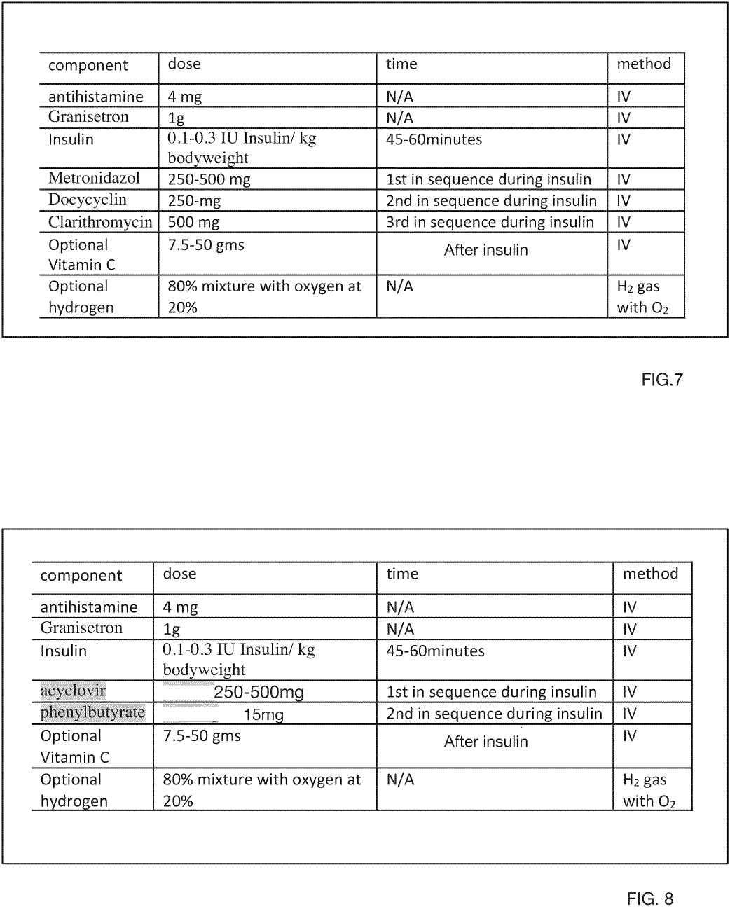

FIG. 3 illustrates an overview of the operational flow of the control system 200. Baseline or nominal values for a patient are collected by way of using a predetermined quantity of a known quality sugar in a predetermined form with a known GL 202. During a time, interval 204 the patient’s blood glucose levels are measured 206 by way of a blood glucose monitoring device (which are known in the art) and the measurements are collected and stored in a database 208. Prior to treating the patient with the disclosed cocktail under hypoglycemic conditions, the patient consumes once again the predetermined quantity of a known quality sugar in a predetermined form with a known GL 210. A time interval 212 will pass after ingestion of the passes after ingestion of the known quality sugar in a predetermined form with a known GL 210 and the previously measured metabolism by the patient of the known quality sugar in a predetermined form with a known GL 202 is collected and can be used to set a hypoglycemic target range that is individualize for the patient. In some instances, the target range for example may be below 54 mg/dl and above 42 mg/dl and with a lower threshold of 40 mg/dl over a predetermined amount of time. In other instances, the target range for example may be below 45 mg/dl and above 38 mg/dl and with a lower threshold of 35 mg/dl over a predetermined amount of time. Based on the metabolic data collected from the patient prior to treatment the controller can individualize the response to allow a lower threshold based on the time a patient can be at that lower threshold before changing the infusion rate of insulin or adding glucose. The system is predictive and can use the slope of the curve of BGL during treatment to reduce insulin or add glucose prior to the patient falling below a lower threshold. In general, 30 mg/dl is a second threshold that should not be maintained, and the patient should not drop below that threshold. However, based on sensor data that level may be raised for a patient.

The controller starts the infusion of insulin 214, the controller starts the infusion of cocktail and/or adjuvant 216 and the sensors 110 measure the patient’s GBL and measures vital signs 218. The skilled artisan or those of ordinary skill in the art will recognize that the sequence of infusing cocktail components, adjuvants, and the like before insulin or vice versa and/or any time gap between the infusions are variations of the disclosed process which are within the scope of this disclosure. A monitoring module 250 receives the measurements and the controller decisions if threshold levels are met for one or more of blood glucose levels (BGL) 262, oxygen 264 and vitals 266. If all threshold levels being monitored are met the then the controller continues the insulin infusion and cocktail / adjuvant infusions, and the system goes on to the timer module 275. If the BGL threshold 262 is not met then the controller will one or more of adjust insulin infusion, add glucose and alert via an alarm. If the O₂ threshold 264 is not met then the controller will one or more of adjust flow rate of the O₂ 150 delivered to patient, adjust one or more cocktail components 130, adjust infusion of one or more supplement / adjuvant components 140 flow rates, adjust other gaseous flow 160 and alert via an alarm. If the vitals threshold 266 is not met then the controller will one or more of adjust insulin infusion, adjust cocktail and/or adjuvant components infusion rates, add glucose and alert via an alarm. If threshold were not met then the sensors 110 measurements of the patient’s blood glucose levels and measure of vital signs 218 are processed by the controller and the controller in the monitoring module 250 as described above decisions if the threshold are been met the monitoring and adjustments repeat.

In another exemplar, prior to treating the patient with the disclosed cocktail under hypoglycemic conditions, the patient consumes once again the predetermined quantity of a known quality sugar in a predetermined form with a known GL 210. A time interval 212 will pass after ingestion of the passes after ingestion of the known quality sugar in a predetermined form with a known GL 210 and the previously measured metabolism by the patient of the known quality sugar in a predetermined form with a known GL 202 is used at least in part by the controller to adjust insulin levels during the treatment. The controller starts the infusion of insulin 214, controller starts the infusion of one or more cocktail components 216 and the sensors 110 measure the patient’s blood glucose levels and measures vital signs 218.

A monitoring module 250 receives the measurements and the controller decisions if threshold levels are met for one or more of blood glucose levels (BGL) 262, oxygen 264 and vitals 266. If all threshold levels being monitored are met the then the controller continues the insulin infusion, and the system goes on to the timer module 275. If the BGL threshold 262 is not met then the controller will one or more of adjust insulin infusion, add glucose and alert via an alarm. If the O₂ threshold 264 is not met then the controller will one or more of the oxygen flow rate 150, adjust gaseous flow rate 160 of the O₂ delivered to patient, adjust one or more cocktail components 130, adjust infusion of one or more supplement / adjuvant components 140 flow rates and alert via an alarm. If the vitals threshold 266 is not met then the controller will one or more of adjust insulin infusion, adjust one or more of the cocktail and/or adjuvant infusion flow rates, add glucose and alert via an alarm. If threshold were not met then the sensors 110 measurements of the patient’s blood glucose levels and measure of vital signs 218 are processed by the controller and the controller in the monitoring module 250 as described above decisions if the threshold are been met the monitoring and adjustments repeat. If threshold levels were met and insulin was continued then the system controller goes to the timer module 275. First elapsed infusion time is measured 278, if the time threshold is not met the measurements of one or more of BGL, O₂ saturation and vital signs are taken 280 and the controller in monitoring mode 250 processes the measurements and repeats the cycle. If the timer has met the threshold 300 the infusion of insulin is stopped and the infusion of cocktail components and/or adjuvant components 302 is stopped and the system measures one or more of BGL, O₂ saturation and vital signs 310 and the controller in timer mode 250 processes the measurements 350 to determine if the post insulin levels of one or more of BGL, O₂ saturation and vital signs are met. If “yes”, then the system stops. If “no” the system one or more of activates alarm and administers one or more of O₂ and glucose to the patient.

FIG. 4 illustrates aspect of controller control logic for a system and method to control hypoglycemic conditions in a primate. It is an overview of some aspects of the operational flow of the control system 300. After the start 301 the controller is in the initial state 302 and will input data from at least on LUT and when available will input data from the primate’s measured baseline 306. The input data is compared with real time sensor inputs 308 and analyzed against predefined threshold or limits and/or target BGL range setting 310. The analysis continues in the monitoring module 250 (described in detail with reference to FIG. 3) wherein the control can adjust the state of the primate by adjusting one or more of glucose, insulin, oxygen, and cocktail components being administered to main or keep the primate in the controlled state (above threshold levels and within target range) and then enter timer module 275 if the timer has timed out end 320.

### EXAMPLES

Cocktail agents for treating bacteria or virus in all instances, are use of controlled hypoglycemic conditions which support the use of lower toxicity cocktails and/or can enter the synovium more easily. Examples listed herein are not intended to be limiting. But rather, a solution of the disclosed delivery system is that the controlled induced hypoglycemic state improves delivery rates of cocktail and/or adjuvants to some bacterial and viruses.

Cocktail agents for treating bacteria or virus in all instances, are use of controlled hypoglycemic conditions which support the use of lower toxicity cocktails and/or can enter the dorsal root ganglion more easily. Examples listed herein are not intended to be limiting. But rather, a solution of the disclosed delivery system is that the controlled induced hypoglycemic state improves delivery rates of cocktail and/or adjuvants to some bacterial and viruses.

Exemplars of cocktail agent antibiotics useful for treating Borrelia Burgdorferi include, but are not limited to clarithromycin, docycyclin, metronidazol. mezloxillian, piperacillin, azlocillin acylampicillin, amoxicillin, cefuroxime, and Ceftriaxone.

Exemplars of cocktail agent antibiotics useful for treating herpes simplex virus (HSV) include, but are not limited to Acyclovir, Famciclovir, Valacyclovir and phenylbutyrate (PBA).

### Examples of Cocktail or Adjuvant Componenets

One animal study provides evidence that the drug azlocillin completely kills off the disease-causing bacteria Borrelia burgdorferi. The study suggests it could also be effective for treating patients infected with drug-tolerant bacteria that may cause lingering symptoms (Pothineni, V.R., Potula, HH.S.K., Ambati, A. et al. Azlocillin can be the potential drug candidate against drug-tolerant Borrelia burgdorferi sensu stricto JLB31. Sci Rep 10, 3798 (2020). https://doi.org/10.1038/s41598-020-59600-4).

Quercetin is poorly soluble in water and unstable in physiological systems, and its bioavailability is very low. Quercetin is a flavonoid widely present in plants and has demonstrated pharmacological properties, including anticancer and senolytic. Quercetin analogs have been studied to increase the low availability of Quercetin, see FIG. 5. Quercetin has also been shown to have antioxidant and anti-inflammatory effects. Lyme disease has inflammation associated with it.

Curcumin has been shown to Curcumin demonstrates significant benefits in the alleviation of arthritic inflammation and gut and brain inflammation. Curcumin exerts anti-inflammatory effects by blocking the activation of NF-κB, a pro-inflammatory signaling pathway heavily involved in Lyme-induced inflammation. Curcumin is a natural compound extracted from the turmeric (see FIG. 6). Curcumin is a potent compound with various biological properties shown to affect HSV-1 IE gene expression which diminishes the ability of the virus to launch the lytic infectious cycle. However, its use as an anti-HSV-1 therapeutic agent has been limited by its low bioavailability. Administration of curcumin is difficult due to its low aqueous solubility, poor oral bioavailability, combining them with micelles or liposomes may ameliorate some of this low availability or by adding adjuvants. Curcumin may be solubilized in a number of ways including but not limited to using a solvent such as DMSO or ethanol, loading curcumin into one of a micelle, nanoparticle or liposome. In some instances, we have included 500 mg of curcumin in a 50ml aqueous solution with 95% total curcuminoid content having 71% curcumin, in DMSO with Kolliphor HS 15 (also known as Macrogol 15 Hydroxystearate, Polyoxyl 15 Hydroxystearate) sodium citrate use only after dilution at least 1: 10. The hydrophobic nature of curcumin presents challenges for bioavailability. A liposome with a mean particle size of about 200 nms composed of dipalmitoylphosphatidylcholine (Lipoid GMBH, Germany) and cholesterol (Carbogen 134 Amcis B.V., The Netherlands) acts as a vehicle to deliver the curcumin at between about 250 mg and about 500 mg. In some instances, the curcumin may be ingested. In other instances, curcumin may be intravenously administered and can also be provided with a hydrophilic carrier.

We have applied a cocktail approach during hypoglycemic conditions of sequenced pharmaceutically effective doses of cocktail compounds to treat infections. Treatment includes IV administration of 0.1-0.3 IU Insulin/ kg bodyweight followed by a sequenced IV administration of multiple chemotherapeutic agents based on the recommendations listed in the oncologic guidelines.

The amount of insulin administered can be a function of pretreatment. When a patient consumes a known quality sugar in a predetermined form with a known glycemic Index (GI) and a known glycemic load (GL) measurement of the patient’s innate systems response to the consumed material can be measured via blood glucose monitoring. Thereafter the controller uses the previously acquired measurements to set target or threshold blood glucose levels to adjust for during controlled hypoglycemic treatment. In operation the patient consumes the same known quality sugar in a predetermined form with a known glycemic Index (GI) and a known glycemic load (GL) at a predetermined time before the controlled hypoglycemic treatment. This system and method personalizes the hypoglycemic process to an individual thereby reducing the risk of insulin shock and seizure.

In general, with our system when the blood glucose level is dropping in a controlled and monitored fashion we are able to have efficacious treatment with approximately 5-10% of the recommended dose of chemo over the same course of 45-60 minutes. The system disclosed herein is configured to sample the sensor data to closely monitor the patient. Before, during and after the system monitors hypoglycemic inducement measuring at least one of EKG, EEG, heart rate, blood pressure, oxygen saturation, glucose levels, pupillary response, temperature, electro galvanic skin resistance / response. The Galvanic Skin Response (GSR), also named Electrodermal Activity (EDA) and Skin Conductance (SC), is the measure of the continuous variations in the electrical characteristics of the skin, i.e., for instance the conductance, caused by the variation of the human body sweating. Sweating is correlated to the effect of the insulin and an indication of treatment progression and status. Optionally at least one of Vitamin C may be added to the IV, hydrogen gas, oxyhydrogen may be administered during or following the controlled hypoglycemic condition as a means to reduce inflammation in general. Although not listed in the flowing tables additional supplements and adjuvants may be included in the cocktail.

We have applied a combinatorial or cocktail approach of sequenced pharmaceutically effective doses of compounds to treat Lyme. We have observed that patients treated with an antihistamine (such as 4 mg Histakut) administered to also reduce cortisol has been observed in our treatment to reduce the effective dose of insulin required to treat. The antihistamine is followed by up to 1gm Granisetron (to relieve nausea) and then an IV of 0.1-0.3 IU Insulin/ kg bodyweight followed by a sequenced IV administration of three antibiotics. The antibiotic regime starts with 250-500 mg Metronidazol then 250 Docycyclin then 500 mg Clarithromycin to be sequence over about 45-to about 60 min followed by 250 cc Glucose 40% and also a glucose-based fluid or juice such Coke and/or Apple juice at 500 cc orally. The treatment is repeated 6-8 times over 2 months and the Borrelia and symptoms are reduced or eradicated. Those of ordinary skill in the art will recognize that antibiotics similar to Metronidazol, Docycyclin and Clarithromycin may be utilized in this treatment regime / sequence without departing from the scope of this invention and expect similar results under said hypoglycemic conditions and as such are within the scope of the disclosure. In some instances, azlocillin may be applied as the active agent in the cocktail alone or in combination with one or more of Metronidazol, Docycyclin and Clarithromycin. The mechanism includes insulin causing the membranes of cells to become more permeable, to allow for energy (glucose) in, the permeability is critical for getting the pharmaceutically effective compounds to the area the borreliosis is aggregated, those areas may include synovial membranes and extracellular matrices. In some instances, the borreliosis has been known to reside in the cerebral spinal fluid as set forth in the table shown in FIG. 7.

As a method, composition of matter, device, and system for treating HSV we have set forth in the table shown in FIG. 8. Those of ordinary skill in the art will recognize that antivirals agents which are in the same class as Acyclovir, Famciclovir, Valacyclovir and phenylbutyrate (PBA) may be substitute and expect similar results under said hypoglycemic conditions. It is preferable that Acyclovir is combined with phenylbutyrate (PBA). A combination of PBA with acyclovir cells infected with HSV-1, the drug combo was able to completely clear the virus from the cells faster and better than either drug alone. Acyclovir is also known to have toxic side effects in the kidneys. (Yadavalli, Tejabhiram & Suryawanshi, Rahul & Koganti, Raghuram & Hopkins, James & Ames, Joshua & Koujah, Lulia & Iqbal, Aqsa & Madavaraju, Krishnaraju & Agelidis, Alex & Shukla, Deepak. (2020). Standalone or combinatorial phenylbutyrate therapy shows excellent antiviral activity and mimics CREB3 silencing. Science Advances. 6. eabd9443. 10.1126/sciadv.abd9443.)

The examples of treatment in tables presented are not intended to be limiting and are nonlimiting examples of a few of the variety of cocktail components, including those that are toxic at the normally used dosages for treating bacteria or viruses, altering the cocktail sequence is within the scope of this disclosure. The exemplary implementations disclosed herein which can be used in provide for greater efficacy of cocktail components at low dose. In some instances, a cocktail component which can be toxic to a patient at the full recommended effective dosage are administered at less than 50% of the normal effective dosage and retain efficacy. In some instances, a cocktail component which can be toxic to a patient at the full recommended effective dosage can be administered at less than 40% of the normal effective dosage and retain efficacy. In some instances, a cocktail component which can be toxic to a patient at the full recommended effective dosage can be administered at less than 30% of the normal effective dosage and retain efficacy. In some instances, a cocktail component which can be toxic to a patient at the full recommended effective dosage can be administered at less than 20% of the normal effective dosage and retain efficacy. In some instances, a cocktail component which can be toxic to a patient at the full recommended effective dosage can be administered at 10% of the normal effective dosage and retain efficacy.

While the compositions and method have been described in terms of what are presently considered to be the most practical and preferred implementations, it is to be understood that the disclosure need not be limited to the disclosed implementations. It will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. It is the intention, therefore, to be limited only as indicated by the scope of the claims appended hereto.

We claim:

1. A system to induce a hypoglycemic condition within a predetermined blood glucose range for treating infection comprising:
one or more sensors which measure an aspect that is physiological;
one or more fluid flow control devices;
insulin in a vessel in fluid communication with a fluid control device;
glucose in a vessel in fluid communication with a fluid control device;
at least one cocktail containing at least one of an antibiotic and an antiviral components in one or more vessels each vessel in fluid communication with a fluid control device;
one or more controllers in signal communication with;
at least one microprocessor;
a blood glucose level (BGL) measuring sensor;
memory;
said one or more sensors;
one or more databases or lookup tables;
said fluid control devices; and,
wherein the controller controls the fluid control devices for at least insulin glucose, and the cocktail to keep blood glucose level (BGL) of the primate within a target hypoglycemic BGL range for the primate.

2. The system to induce a hypoglycemic condition within a predetermined blood glucose range for treating infection of claim 1, wherein the controller receives sensor data inputs and adjust the hypoglycemic target range for BGL in response to sensory data received.

3. The system to induce a hypoglycemic condition within a predetermined blood glucose range for treating infection cells of claim 2, wherein the sensor data is one of BGL, oxygen saturation, heart rate, blood pressure, galvanic skin response, temperature, EEG, ECG, and pupillary response.

4. The system to induce a hypoglycemic condition within a predetermined BGL range for treating infection of claim 3, wherein the controller controls the administration of at least one of oxygen and hydrogen.

5. The system to induce a hypoglycemic condition within a predetermined blood glucose range for treating infection of claim 3 wherein the cocktail is at least one of, Quercetin, curcumin, vitamin C, clarithromycin, docycyclin, metronidazol. mezloxillian, piperacillin, azlocillin acylampicillin, amoxicillin, cefuroxime, Ceftriaxone, Acyclovir, Famciclovir, Valacyclovir and phenylbutyrate (PBA).

6. The system to induce a hypoglycemic condition within a predetermined blood glucose range for treating infection of claim 5, wherein the infection is herpes simplex virus, and the cocktail is at least one of Acyclovir, Famciclovir, Valacyclovir and phenylbutyrate (PBA).

7. The system to induce a hypoglycemic condition within a predetermined blood glucose range for treating infection of claim 5, wherein the infection is B. burgdorferi and the cocktail is at least one clarithromycin, docycyclin, metronidazol. mezloxillian, piperacillin, azlocillin acylampicillin, amoxicillin, cefuroxime, Ceftriaxone.

8. The system to induce a hypoglycemic condition within a predetermined blood glucose range for treating infection of claim 6 or 7, wherein the active agents in the cocktail are each less than 50% the maximum tolerated dose (MTD).

9. The system to induce a hypoglycemic condition within a predetermined blood glucose range for treating infection of claims 6 or 7, wherein the active agents in the cocktail are less than 50% the minimum effective dose (MED).

10. The system to induce a hypoglycemic condition within a predetermined blood glucose range for treating infection of claim 7, wherein the active agents in the cocktail are less than 25% the minimum effective dose (MED).

11. A method to reduce infection in a primate, the method comprising:
inducing a hypoglycemic condition in a primate having infection within a target BGL range by way of infusion, controlled by a controller, of at least insulin;
monitoring vital signs of the primate with one or more sensors each of which monitor a physiological aspect of the primate and are in signal communication with the controller;
controlling with a controller infusion of a cocktail containing at least one of antibiotic and an antiviral components into the primate while the primate is in a hypoglycemic condition;
wherein the controller receives data inputs form the sensors and at least in part uses that input data to one of maintain the primate’s BGL within a hypoglycemic range for BGL and alter the hypoglycemic BGL range or lower threshold for the hypoglycemic BGL based on the received sensor data; and.
wherein cellular membranes of the primate are made more susceptible to the influx of cocktail components by way of the hypoglycemic condition.

12. The method to reduce infection in a primate, of claim 11, wherein the infection is one of HSV and B. burgdorferi.

13. The method to reduce infection in a primate, of claim 11, wherein the sensor data is one of BGL, oxygen saturation, heart rate, blood pressure, galvanic skin response, temperature, EEG, ECG, and pupillary response.

14. The method to reduce infection in a primate, of claim 11, wherein the controller controls the administration of at least one of oxygen and hydrogen.

15. The method to reduce infection in a primate, of claim 11, wherein the infection B. burgdorferi and the is cocktail is at least one of, clarithromycin, docycyclin, metronidazol. mezloxillian, piperacillin, azlocillin, acylampicillin, amoxicillin, cefuroxime, Ceftriaxone.

16. The method to reduce infection in a primate of claims 15, wherein the active agents in the cocktail are each less than 50% the maximum tolerated dose (MTD).

17. The method to reduce infection in a primate of claims 15, wherein the active agents in the cocktail are less than 50% the minimum effective dose (MED).

18. The method to reduce infection in a primate of claims 15, wherein the active agents in the cocktail are less than 25% the minimum effective dose (MED).

19. The method to reduce infection in a primate of claims 15, wherein the active agents in the cocktail are less than 15% the minimum effective dose (MED).

20. The method to reduce infection in a primate of claim 11, the method further comprising the controller raises BGL in the primate by way of infusion of at least glucose to maintain the hypoglycemic range for the primate’s BGL.

21. The method to reduce infection in a primate of claims 20, the method further comprising the controller administers magnesium before or during administration of glucose.
